(19) United States
(12) Patent Application Publication
(10) Pub. No.: US 2020/0038423 A1
(43) Pub. Date: Feb. 6, 2020

(54) USE OF 6-O-(C8-C20 ALKYL ESTER) OF 1-O-(C1-C6 ALKYL)-ß-D-GLUCOSIDE AS AN AGENT FOR PROTECTING THE SKIN

(71) Applicant: PIERRE FABRE DERMO-COSMETIQUE, BOULOGNE-BILLANCOURT (FR)

(72) Inventors: Sylvie DAUNES MARION, TOULOUSE (FR); Marie Florence GALLIANO, BLAGNAC (FR); Hélène HERNANDEZ-PIGEON, CUGNAUX (FR); Stéphane POIGNY, SAUBENS (FR)

(73) Assignee: PIERRE FABRE DERMO-COSMETIQUE, BOULOGNE-BILLANCOURT (FR)

(21) Appl. No.: 16/339,265

(22) PCT Filed: Oct. 5, 2017
(86) PCT No.: PCT/EP2017/075406

§ 371 Date: Apr. 3, 2019

(30) Foreign Application Priority Data
Oct. 5, 2016 (FR) 1659616

(51) Int. Cl. A61K 31/7028; A61K 9/00; A61P 17/00; A61K 8/60; A61Q 19/00

(52) U.S. Cl. CPC A61K 31/7028; A61K 9/0014; A61Q 19/00; A61K 8/602; A61P 17/00

(57) ABSTRACT

The present invention relates to the use of 6-O-(C8-C20 alkyl ester) of 1-O-(C1-C6 alkyl)-β-D-glucoside and in particular 6-O-lauroyl of 1-O-n-butyl-β-D-glucoside as an agent for protecting the skin and/or mucous membranes. The invention relates specifically to a cosmetic, dermatological and/or pharmaceutical composition, intended for topical use, which can be used for skin care and/or treatment.

The present invention concerns the use of 1-O-(C1-C6 alkyl)-β-D-glucoside 6-O-(C8-C20 alkyl ester) and in particular 1-O-n-butyl-β-D-glucoside 6-O-lauroyl as protective agent for the skin and/or mucous membranes. More particularly, it concerns a cosmetic, dermatological and/or pharmaceutical composition, intended for topical use, which can be used for skin care and/or treatment.

These glucoside derivatives are useful as active agents acting on skin hydration, strengthening of the barrier function, maintenance of epidermal homeostasis, resistance to various stresses, cell repair and tissue regeneration.

### Epidermal Homeostasis

The epidermis plays a major protective role as a mechanical and chemical barrier for the body. It ensures that an impermeable skin barrier function is maintained. It is the corneocytes, the keratinocytes of the stratum corneum, together with a lipid matrix, which provide the vast majority of this function. However, the deepest layers also take part in constructing the elements inherent to this function. The ability of epidermal keratinocytes to differentiate results in the construction of a barrier having the selective permeability function (Elias and Choi, Exp. Dermatol. 14(10), p19-26, 2005). Keratinocyte differentiation is regulated in space and time, from the deepest layers of the skin, the basal membrane being the least differentiated, to the stratum corneum or horny layer, the final step of differentiation of keratinocytes into corneocytes (Houben et al., Skin Pharmacol. Physiol. 20(3), p122-132, 2007). From a cellular and molecular point of view, the formation of keratin filaments, the transformation of keratinocytes into corneocytes or “keratinization”, and the formation of an intercellular lipid cement of lamellar structure are mainly observed, ensuring impermeability and the skin barrier function.

In terms of proteins, epidermal differentiation focuses mainly on the development of structural proteins called keratins, which contribute to the architectural integrity of the epidermis. Their expression varies as a function of keratinocyte maturity. Alkaline keratin 1 and acidic keratin 10 are early markers of keratinocyte differentiation, present in the basement membrane of the epidermis. The expression of other markers in this biological process, which occurs later, can be followed in the same way as for the cornified cell envelope, such as involucrin, together with certain major enzymes that cause structural proteins to form bridges with each other and with keratinocyte lipids and transglutaminases, such as TGM1 or 3 (Houben et al., Skin Pharmacol. Physiol. 20(3), p122-132, 2007).

The fibrous matrix present in corneocytes is formed during the transition between granular keratinocytes and corneocytes. Loricrin is a structural protein containing glutamine and lysine residues which allow adhesion to other proteins in the cornified cell envelope. The basic filaggrin molecules produced from their precursor, profilaggrin, stored in keratohyalin granules, combine with cytokeratin filaments and can then aggregate. Filaggrin, degraded by caspase 14, is also the main source of several major components of the natural moisturizing factor in the stratum corneum. Other markers are specific to differentiated keratinocytes. Kallikreins, such as KLK5 and KLK7, have an activity similar to that of chymotrypsin and play a role in the proteolysis of cohesive intercellular structures that precede desquamation, namely the removal of the outermost layer of the epidermis.

At the same time, the synthesis and transport of keratinocyte lipids form the foundation of the intercorneocyte lipid cement, essential to the skin barrier, the formation of which represents the final phase in terminal epidermal differentiation. This extracellular lipid matrix is the main barrier for the transcutaneous transport of fluids and electrolytes (Feingold, J. Lipid Res. 48, p2529-2530, 2007). Thus, a certain number of enzymes and lipid transporters see the expression of their keratinocytes regulated upwards in conjunction with differentiation. The cement results from the equilibrium between three lipid species, namely cholesterol, free fatty acids and ceramides. These lipids come from glucosylceramides, sphingomyelin, cholesterol and phospholipids produced in the stratum spinosum (horny layer) and the stratum granulosum (granular layer). They are transported by lamellar bodies, which are secretory organelles that fuse with the granular layer and the horny layer. In addition to these lipid precursors, lamellar bodies contain many enzymes, including lipidases such as acid sphingomyelinase, beta-glucocerebrosidase and phospholipases A2, together with acid and neutral lipases. Delivered to extracellular spaces at the same time as lipid precursors, these enzymes convert sphingomyelin to ceramide, glucocerebrosides to ceramide and phospholipids to free fatty acids and glycerol, respectively. SULT2B1 is a cholesterol sulfotransferase expressed in differentiated keratinocytes and is involved in the synthesis of cholesterol sulfate. A recent study also found that cholesterol sulfate induces filaggrin expression through increased RORα expression (Hanyu et al., Biochem. Biophys. Res. Common. 428(1), p99-104, 2012).

Epidermal ceramides play a major specific role and represent an essential marker of the level of functionality of the skin barrier. The expression and activity of enzymes involved in the production of skin ceramides specifically increase when the skin barrier function is altered, and conjointly with the level of epidermal differentiation (Feingold, J. Lipid Res. 48, p2529-2530, 2007). This is the specific case of an aSMase and β-glucoceramidase, involved in the extracellular metabolism of skin ceramides. UDP-glucose: ceramide glucosyltransferase (UGCG) is also involved in glucosylceramide synthesis. UGCG catalyzes the first glycosylation step in glycosphingolipid biosynthesis and is necessary for the regular arrangement of lipids and proteins in lamellar bodies and for the maintenance of the epidermal barrier (Jennemann et al., J. Biol. Chem. 282(5), p3083-3094, 2007). DEGS2 acts as both sphingolipid C4-hydroxylase and delta-4 desaturase, its dihydroceramide hydroxylase activity competing with the production of skin phytoceramides in humans (Mizutani et al., FEBS Lett. 563(1-3), p93-97, 2004).

FABP-E (FABP5), an epidermal fatty acid binding protein, is a lipid transporter. FABP-E plays an important role in keratinocyte differentiation (Dallaglio, et al., Exp Dermatol. 22(4), p255-261, 2013).

One of the functions of water in the stratum corneum is to activate enzymatic hydrolysis reactions necessary for skin suppleness and normal desquamation (Rawlings and Matts, J. Invest. Dermatol, 124(6), p1099-1110, 2005). If the water content in the stratum corneum falls below a critical level, enzymatic reactions are disrupted, leading to corneocyte adhesion and cell accumulation on the skin's surface. This creates visible dryness and itching, the skin peels and exfoliates.

The skin's hydration is based on two points, the supply of transepidermal water from the cutaneous blood circulation and the retention of epidermal water, which involves the skin barrier function. However, the barrier to water loss is not infallible. A normal exchange of water between the external and internal environments through the stratum corneum is known as transepidermal water loss (TEWL) and is inherent in insensible water loss (IWL).

### Wound Healing

Wound healing, cell repair and epidermal regeneration combined, is traditionally divided into four phases: hemostasis, inflammation, repair and remodeling (Reinke and Sorg, Eur. Surg. Res., 49, p35-43, 2012). Two to ten days after an injury, there is strong cell proliferation and migration to quickly restore the tissue and its vascularization. During this phase, reepithelialization is the process of covering a wound by regeneration of epidermal keratinocytes. This restores the protective barrier function against the external environment and thus reduces morbidity and mortality following an injury.

Reepithelialization consists of three phenomena, occurring in parallel but staggered over time, in the regenerating epidermis:
keratinocyte migration
keratinocyte proliferation
maturation of the neoepidermis.

Following the rupture of the epidermis, the keratinocytes become activated by undergoing changes in their cytoskeleton. The migrating keratinocytes take the form of a reepithelialization tongue, these migrating keratinocytes do not follow the usual differentiation pattern but remain in an undifferentiated stage expressing keratins of the basal layer (5 and 14) but few proteins characteristic of differentiation. Behind the migrating front, the keratinocytes proliferate. This proliferation, limited to the basal layer, is induced by a combination of growth factors including EGF, TGF-α and GM-CSF. Once the wound is completely epithelialized, the keratinocytes integrate their terminal differentiation program and begin their stratification. The necrotic tissues and the blood clot are pushed back by cells of the hyperproliferating neoepidermis whose superficial layers desquamate.

Thus, keratinocyte migration is an essential process from the first days after an injury to allow regeneration of the epidermis, particularly the neoepidermis, as well as cell repair.

Keratinocyte migration can be evaluated in vitro from primary human keratinocyte cultures using standardized operating techniques such as those developed in the Oris™ Cell Migration Assay (Platypus Technologies-TEBU).

The subject-matter of the present invention concerns at least one 1-O-(C1-C6 alkyl)-β-D-glucoside 6-O-(C8-C20 alkyl ester) compound for use as epidermal regeneration and/or epidermal cell repair agent. Preferably, the subject-matter of the invention concerns 1-O-n-butyl-β-D-glucoside 6-O-lauroyl for use as epidermal regeneration and/or epidermal cell repair agent.

The applicant has shown that a 1-O-(C1-C6 alkyl)-β-D-glucoside 6-O-(C8-C20 alkyl ester) compound and more particularly 1-O-n-butyl-β-D-glucoside 6-O-lauroyl provides skin hydration, strengthens the barrier function of the epidermis, maintains epidermal homeostasis, provides resistance to various stresses. In doing so, the compound according to the invention contributes to epidermal cell repair and/or to epidermal regeneration or to wound healing. This effect could be demonstrated by inducing the expression of mRNA of various genes involved in keratinocyte differentiation and hydration, but also by stimulating keratinocyte migration.

Skin hydration means improving or maintaining the skin's water balance.

Improvement of any form of skin dryness means any improvement in the hydration of the epidermis, especially characterized by a lack of water in the stratum corneum, a too-thin hydrolipidic film located on the surface which no longer protects the skin, the lack of sebum.

The subject-matter of the present invention also concerns a dermatological, cosmetic or pharmaceutical composition comprising, or consisting of, as active principle, at least one 1-O-(C1-C6 alkyl)-β-D-glucoside 6-O-(C8-C20 alkyl ester) compound and at least one acceptable excipient, for use in epidermal regeneration and/or epidermal cell repair.

Preferably, the use is a topical use.

The dermatological, cosmetic or pharmaceutical composition, which comprises as active principle at least one 1-O-(C1-C6 alkyl)-β-D-glucoside 6-O-(C8-C20 alkyl ester) compound and at least one acceptable excipient, is also useful for promoting epidermal cell repair and/or for promoting epidermal regeneration, or for promoting wound healing.

Advantageously, the composition according to the invention is intended for topical application on the skin and skin appendages, the scalp and the mucous membranes.

In a particular embodiment of the invention, 1-O-(C1-C6 alkyl)-β-D-glucoside 6-O-(C8-C20 alkyl ester), more particularly 1-O-n-butyl-β-D-glucoside 6-O-lauroyl, is preferred as active principle in the composition.

More preferably, 1-O-(C1-C6 alkyl)-β-D-glucoside 6-O-(C8-C20 alkyl ester), more particularly 1-O-n-butyl-β-D-glucoside 6-O-lauroyl, is the only active principle of the composition having an effect on epidermal cell repair and/or epidermal regeneration, or on wound healing.

According to the invention, the dermatological or cosmetic composition may comprise from 0.01 to 10%, preferably from 0.1% to 5%, and more particularly from 0.1 to 3% and even more particularly from 0.1 to 2% by weight 1-O-n-butyl-β-D-glucoside 6-O-lauroyl based on the total weight of the composition.

In a preferred embodiment, the composition according to the invention will preferentially be in the form of a skin care and/or make-up product for the body or face, the lips, the eyelashes, the eyebrows, the hair, the scalp, the nails or the mucous membranes, a sunscreen or self-tanning product, a hair product notably for coloring, conditioning and/or caring for the hair.

Topical administration of the dermatological or cosmetic composition may be accomplished by the application of a solution, suspension, aqueous gel, lotion, serum, milk, salve, ointment, cream, eye drops, or other vehicle used for topical application, well known to the skilled person, notably in hydro-alcoholic form, the form of an oil-in-water or water-in-oil or multiple emulsion, an oil gel, or an anhydrous liquid, pasty or solid product or in dispersion form in the presence of spherules. These compositions are prepared according to the usual methods. One of the possible means is the administration of the dermatological or cosmetic composition by an aerosol spray allowing fine liquid droplets to be vaporized for distribution over the entire surface necessary or, conversely, precisely limited to a particular target zone, or in solid form, in stick form. Another exemplary embodiment is the patch that provides continuous release of the topical composition. The patch may have a reservoir and a porous membrane or a solid matrix, which are well known to the person skilled in the art.

The oils usable in the invention are those generally used in the fields concerned. They may be vegetable, mineral or synthetic oils, optionally silicone and/or fluorinated oils.

The topical compositions according to the invention may also contain hydrophilic or lipophilic adjuvants such as gelling agents, preservatives, opacifiers, emulsifiers, co-emulsifiers, neutralizers, fragrances, and their solubilizers or peptizers, dyes, pigments, antiseptics, antioxidants, mineral salts, thickeners, pH modifiers, ultraviolet ray absorbers, vitamins or any other cosmetically and dermatologically acceptable excipient well known to the skilled person.

The composition according to the invention may further contain another topically active agent, or a mixture of such active agents.

Surfactant means, in the traditional sense, any amphiphilic molecule capable of acting on the interfacial tension of a dispersed medium. The composition according to the invention is thus free of such a surfactant able to solubilize the hydrolipidic film of the skin. Advantageously, it may also be free of preservatives, which may be responsible for skin intolerances, especially any quaternary ammonium, ethanol, phenols, amidines, isothiazolone derivatives, parahydroxybenzoic esters (known as parabens), etc.

The compositions described can be applied to the surface of the patient's skin to be treated. The frequency of application will depend on the circumstances and the person. For example, the compositions may be applied daily, twice a day, or even more frequently.

A glucoside consists of a sugar (typically a monosaccharide) linked to a non-carbohydrate substance by an O-bond. In particular, alpha (or α) glucosides are distinguished from beta (or β) glucosides. Beta-glucosides have a glycosidic bond of beta form, which is a covalent chemical bond between the reducing group (hydroxyl) of the alcohol function of the hemiacetalic carbon of a monosaccharide (anomeric carbon, number 1 in aldoses such as glucose) and the acid group (free hydrogen) of another molecule.

The present case relates to an alkyl D glucoside of beta form. It is a glucoside involving D glucose whose alcohol function of the hemiacetal carbon 1 in beta conformation has been condensed with an alkyl chain molecule having a hydroxyl, here a fatty chain alcohol which is therefore in position 1.

As for the alkyl ester moiety of the molecule, it is the esterification with an alkyl compound having a carboxylic residue of the secondary alcohol of glucose, i.e. in position 6.

The terms C8-C20 alkyl and C1-C6 alkyl refer to linear or branched hydrocarbon residues having between 8 and 20 carbon atoms and between 1 and 6 carbon atoms, respectively.

Another subject-matter of the invention concerns a process for the stereoselective preparation of 1-O-(C1-C6 alkyl)-β-D-glucoside 6-O-(C8-C20 alkyl ester), and in particular 1-O-n-butyl-β-D-glucoside 6-O-lauroyl. This process involves the following successive steps:

a) acetalization of D-glucose with a C₁-C₆ alkanol in the presence of a hydrolase enzyme to form the corresponding 1-O-(C1-C6 alkyl)-β-D-glucoside;

b) esterification of the 1-O-(C1-C6 alkyl-β-D-glucoside obtained at the end of step a) with a C₈-C₂₀ fatty acid in the presence of a lipase enzyme to form said 1-O-(C1-C6 alkyl)-β-D-glucoside 6-O-(C8-C20 alkyl ester).

The β-LBG obtained by the process described above has unexpectedly improved dermo-cosmetic properties due to their specific form of purity.

The following examples perfectly illustrate the present invention without limiting its scope.

### EXAMPLE 1: EVALUATION OF 1-O-N-BUTYL-β-D-GLUCOSIDE 6-O-LAUROYL ON the Modulation of Genes Involved in Keratinocyte Differentiation and Hydration

Normal human keratinocytes were incubated for 48 h with 1-O-n-butyl-β-D-glucoside 6-O-lauroyl. Its effects were evaluated using the RT-qPCR technique with the analysis of 6 target genes, chosen for their importance in keratinocyte differentiation and hydration.

The results of this study are summarized in Table 1 below.

| | | | | 1-O-n-butyl-β-D- | |
|---|---|---|---|---|---|
| | | | | glucoside 6-O- | |
| | | CaCl2 | | lauroyl | |
| | | 1.5 mM | | 2 0 μM | |
| | | Mean | | Mean | |
| Genes | | (% Control) | SD | (% Control) | SD |
| Lipid | SULT2B1 | 1169 | 25 | 549 | 122 |
| differentiation | FABP5 | 501 | 179 | 259 | 1 |
| | DEG S2 | 217 | 125 | 277 | 16 |
| Protein | KLK7 | 994 | 220 | 253 | 43 |
| differentiation | TGM1 | 910 | 89 | 536 | 97 |
| | CASP14 | 127 | 50 | 441 | 25 |

SULT2B1: Sulfotransferase family, cytosolic, 2B, member 1;
FABP5: Fatty acid binding protein 5;
DEGS2: Degenerative spermatocyte homolog 2, lipid desaturase;
KLK7: Kallikrein-related peptidase 7;
TGM1: Transglutaminase 1;
CASP14: Caspase 14.

Under the analytical conditions, it was demonstrated after incubation for 48 h that 1-O-n-butyl-β-D-glucoside 6-O-lauroyl at 20 μM induces reproducible keratinocyte differentiation. Indeed, at this concentration, 1-O-n-butyl-β-D-glucoside 6-O-lauroyl induces the expression of the lipid differentiation markers SULT2B1, FABP5 and DEGS2. It thus participates in the sulfonation of cholesterol and the synthesis of fatty acids and ceramides. 1-O-n-butyl-β-D-glucoside 6-O-lauroyl also induces the protein markers of differentiation KLK7, involved in desquamation, transglutaminase 1 (TGM1) and caspase 14 (CASP14), involved in the degradation of filaggrin into a natural moisturizing factor.

1-O-n-butyl-β-D-glucoside 6-O-lauroyl therefore plays a very important role in keratinocyte differentiation and provides the protein and lipid physical barrier function. It thus restores and strengthens the barrier function, regenerates the epidermis and repairs cells, and prevents skin dehydration.

### EXAMPLE 2: EVALUATION OF 1-O-N-BUTYL-β-D-GLUCOSIDE 6-O-LAUROYL ON KERATINOCYTE MIGRATION

The purpose of the study described in this example is to evaluate the effects of 1-O-n-butyl-β-D-glucoside 6-O-lauroyl on the migration of primary normal human keratinocytes.

Oris™ Cell Migration Assays were used to estimate cell migration.

Primary normal human keratinocytes isolated from tissues extracted during cosmetic surgery were seeded on 96-well plates and cultured in KSFM supplemented with 25 μg/ml bovine pituitary extract (BPE, Invitrogen) and 0.2 ng/ml EGF (Invitrogen). After 24 h of incubation, the culture medium is replaced with KSM without supplement. After 24 h of incubation, the Oris™ stoppers as well as the test compounds or positive controls are removed. The latter are 5 ng/ml TGF-β, 30 ng/ml EGF, or KSM supplemented with BPE and EGF. Incubation continues for the next 24 h to allow migration. At the end of incubation, Calcein AM dye, which penetrates the cells, is added to the wells and is converted to fluorescence which is then quantified in a detection zone using a monochromatic microplate (Clariostar, BMG Labteck).

The compound tested is 1-O-n-butyl-β-D-glucoside 6-O-lauroyl, it is solubilized in DMSO at a concentration of 100 mM. The compound is tested at 2, 10 and 20 μM. The effect of DMSO at the tested concentrations has no effect on keratinocyte migration. The experiments are repeated on 2 donors with 8 wells for each condition.

The quantification of the fluorescence emitted in the detection zone is proportional to keratinocyte migration. Statistical analyses (Mann-Whitney test) are used to analyze the effect of the compound. After fluorescence quantification, the cells were rapidly fixed in 4% paraformaldehyde before eosin-hematoxylin staining and observed under a microscope.

The results of this study are summarized in Table 2 below:

| | % Stim | | | | | | |
|---|---|---|---|---|---|---|---|
| | | Positive controls | | | | | |
| | Neg | KSFM | | | | | |
| | Ctrl | EGF¹ | TGF | EGF | β-LBG | | |
| Donors | KSFM | BPE² | 5 ng/ml | 30 ng/ml | 2 μM | 10 μM | 20 μM |
| 1 | 0 | 170 | 266 | 35 | −30 | 41 | 104 |
| | | * | ** | | | | ** |
| 2 | 0 | 27 | 14 | 87 | 46 | 63 | 21 |
| | | | | * | | * | |

% Stim: % stimulation; Neg Ctrl: negative control; β-LBG: 1-O-n-butyl-β-D-glucoside 6-O-lauroyl.
EGF¹: 0.2 ng/ml; BPE²: 25 μg/ml.
* p < 0.05;
** p < 0.01.

The positive controls show strong stimulations of migration (87% to 266%) thus validating these experiments.

Unexpectedly, the inventors show that 1-O-n-butyl-β-D-glucoside 6-O-lauroyl stimulates keratinocyte migration on both donors tested at either 10 μM or 20 μM, with a higher stimulation obtained at 20 μM in the first donor and at 10 μM in the second donor.

In conclusion, by using the OrisT Cell Migration Assay on normal human keratinocytes, 1-O-n-butyl-β-D-glucoside 6-O-lauroyl in concentrations of 10 to 20 μM significantly stimulates the migration of normal human keratinocytes and thus contributes to epidermal regeneration and epidermal cell repair.

1. Use of at least one 1-O-(C1-C6 alkyl)-β-D-glucoside 6-O-(C8-C20 alkyl ester) compound as epidermal regeneration and/or epidermal cell repair agent.

2. Use according to claim 1, characterized in that said compound is 1-O-n-butyl-β-D-glucoside 6-O-lauroyl.

3. Dermatological or cosmetic composition, characterized in that it comprises as active principle at least one 1-O-(C1-C6 alkyl)-β-D-glucoside 6-O-(C8-C20 alkyl ester) compound and at least one dermatologically or cosmetically acceptable excipient, for use in epidermal regeneration and/or epidermal cell repair.

4. Dermatological or cosmetic composition according to claim 3, characterized in that the amount of 1-O-(C1-C6 alkyl)-β-D-glucoside 6-O-(C8-C20 alkyl ester) varies between 0.01% and 10% by weight based on the total weight of the composition.

5. Dermatological or cosmetic composition according to one of claims 3 or 4, characterized in that the active principle is 1-O-n-butyl-β-D-glucoside 6-O-lauroyl.
